(19) United States
(12) Patent Application Publication
(10) Pub. No.: US 2003/0180800 A1
(43) Pub. Date: Sep. 25, 2003

(54) Stable isotope based dynamic metabolic profiling of living organisms for characterization of metabolic diseases, drug testing and drug development

(76) Inventors: Wai-Nang Paul Lee, Palos Verdes Estate, CA (US); Laszlo G. Boros, Torrance, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/192,744

(22) Filed: Jul. 9, 2002

Related U.S. Application Data
(60) Provisional application No. 60/367,142, filed on Mar. 22, 2002.

(51) Int. Cl. C12N 13/00

(52) U.S. Cl. 435/7.1; 435/173.8

(57) ABSTRACT

The metabolic processes involved in the formation of any glucose-based metabolite are determined. A precursor molecule is labeled with a stable carbon (¹³C) isotope at specific positions. The label is allowed to distribute and rearrange in the system. Metabolites are recovered and analyzed against a control system to determine metabolic pathway substrate flux caused by changes to the test system relative to the control system such as the addition of compound being tested as a potential drug.

### CROSS-REFERENCE

This application claims the benefit of U.S. Provisional Application No. 60/367,142, filed Mar. 22, 2002, which application is incorporated herein by reference.

### GOVERNMENT RIGHTS

This invention was made with government support under federal grant nos. P01CA42710-15 awarded by National Institutes of Health to UCLA-CNRU. The United States Government may have certain rights in this invention.

### FIELD OF THE INVENTION

This invention relates generally to the field of biochemical methodologies. The invention further relates to the field of stable (¹³C) isotope labeling of a metabolome to examine mechanisms of cellular substrate flow modifications in response to various drugs, food additives, natural compounds and environmental factors to obtain information such as how they affect cellular physiology based on metabolic pathway substrate flow, intermediate production and end-product synthesis.

### BACKGROUND OF THE INVENTION

Metabolic profiling or metabolomics is an old investigative field where the amounts or concentrations of various metabolites of various pathways in living organisms are measured and, from these determinations, activities of the respective metabolic pathways are predicted (Katz, J., Rognstad, R. (1967). Specific examples include the labeling of pentose phosphate from glucose-¹⁴C and estimation of the rates of transaldolase, transketolase, the contribution of the pentose cycle to ribose phosphate synthesis. Biochemistry 6: 2227-47). In general, these techniques only provide information on a static picture of a cell at one point in time and only measure synthesis rates without being able to reveal specific reactions and their contributions to end-product synthesis. The technique does not exactly reveal the previous metabolic steps and the exact synthesis pathways but only estimates the involvements of possible metabolic pathways based on existing biochemical information.

There are many alternative pathways throughout cellular metabolism to produce various metabolites which may make it difficult to elucidate particular enzymatic reactions using static metabolic profiling (Raamsdonk, L. M., Teusink, B., Broadhurst, D., Zhang, N., Hayes, A., Walsh, M. C., Berden, J. A., Brindle, K. M., Kell, D. B., Rowland, J. J., Westerhoff, H. V., van Dam, K., Oliver, S. G. (2001). A functional genomics strategy that uses metabolome data merely to reveal the phenotype of silent mutations Nat Biotechnol 19: 45-50) generally does not reveal substrate flow and enzymatic substrate modifications in interconnected and complex metabolite networks.

Leading laboratories in stable isotope based metabolite research use single labeling patterns and measure single pathways in mammalian cells in order to reveal specific synthesis steps of bio-molecules. These pathways may be involved in cell proliferation (Neese, R. A., Siler, S. Q., Cesar, D., Antelo, F., Lee, D., Misell, L., Patel, K., Tehrani, S., Shah, P., Hellerstein, M. K. (2001). Advances in the stable isotope-mass spectrometric measurement of DNA synthesis and cell proliferation have also been described Anal Biochem 298: 189-95). However these methods generally measure new cell production through DNA synthesis without the specifics of metabolic pathway activities and their contribution to the cellular proliferation process. Further, others have carried out work applied to gluconeogenesis (Previs, S. F., Brunengraber, H. (1998) to measure the production of glucose in vivo (Curr Opin Clin Nutr Metab Care 1: 461-5), as well as de novo lipid and fatty acid synthesis (Verhoeven, N. M., Schor, D. S., Previs, S. F., Brunengraber, H., Jakobs, C. (1997).

Stable isotope studies of phytanic acid alpha-oxidation and in vivo production of formic acid has also been described (Eur J Pediatr 56: S83-7). Stable isotopes are also used as standards for quantification of known compounds in the blood and body fluids (Leis, H. J., Windischhofer, W., Raspotnig, G., Fauler, G. (2001) and others have described stable isotope dilution negative ion chemical ionization gas chromatography-mass spectrometry for the quantitative analysis of paroxetine in human plasma (J Mass Spectrom 36: 923-8; Andrew, R. (2001) as well as the clinical measurement of steroid metabolism (Best Pract Res Clin Endocrinol Metab 15: 1-16). Although important for the quantitation of metabolite synthesis and turnover rates, these papers generally do not attempt to analyze the metabolome, as a whole, by its selected and representative components synthesized through individual metabolic reactions, which are linked, interconnected and are capable of cross-label the cellular intermediary metabolite pool as they rearrange and re-distribute ¹³C labeled substrate carbons from one stable isotope labeled precursor, which, in turn, imprints a metabolic “history” and “memory” into the dynamically formed product pool throughout the life cycle of the organism and drug treatments.

### SUMMARY OF THE INVENTION

The stable ¹³C isotope based glucose substrate [1,2-¹³C₂]glucose readily and dynamically labels intracellular metabolic pathways through active metabolic steps resulting in a stable isotope labeled metabolome. This stable isotope enriched metabolome contains substrates and products, which reveal synthesis patterns, destinies and distributions of the labeled glucose among major metabolic pathways broadly. This technique is utilized for drug target discovery, and for testing and screening of compounds, which may be used as pharmaceutically active drugs, food additives, natural products with physiological activities or changes in cellular environment. The metabolic effects of new compounds such as drugs, as specifically tested by ¹³C labeled glucose, reveal the metabolic end result of genetic manipulations and cell-signaling events because changes in the stable isotope labeled metabolome closely reflect changes in metabolic enzyme activities that are primarily controlled by genes or protein phosphorylating signals. The metabolome can provide information on changes in cellular function in a chain of genetic, proteomic and metabolic events in a living organism. As the genome and proteome already have their own labeling technologies and techniques, it is evident that comprehensive studies of the metabolome will also require a broad, effective, yet specific label system for drug studies to come.

The ¹³C labeled glucose substrate is provided to a system which may be cells in a cell culture. The cells are used to create an information profile which details any desired aspect(s) of cellular metabolism including metabolic pathway substrate flow, specific metabolite synthesis patterns, rate of metabolite synthesis, contribution of individual synthetic reaction, etc. Once the information profile is created labeled glucose can be added to a substantially identical system to which is added a compound such as a drug to be tested. The information profile created in the absence of the drug is then compared to a new information profile created with the drug in the system. Other parameters such as the concentration of the drug added to the system and/or the amount of time allowed to pass can be changed to obtain different information profiles which when analyzed provide information on how the drug effects the system on a molecular metabolic level. This allows the testing of new drug candidates in a dose and time dependent with information on their metabolic effects, toxicity and regulatory mechanisms on metabolic pathway substrate flow, cell function and phenotype.

The invention uses a label such as a non-toxic stable ¹³C labeled glucose isotope in a unique way. The methodology is applied for characterizing the complex dynamic metabolic profiles of diseases and to investigate the mechanism of action of new and existing compounds and in particular therapeutic compounds. The invention may enhance the ability for discovering new drug target sites through metabolic enzymes, which strongly and effectively control substrate flow and distribution in living organisms. This technique can also be used in the drug industry to reveal the exact mechanism of action of new drugs on metabolism and to reveal toxicity, which will accelerate the drug testing, candidate selection and drug approval processes.

The understanding of drug actions and the mechanisms of diseases is assisted by the characterization of metabolic pathways through the flow of substrates. Genes and signal transduction pathways can only trigger changes in cellular metabolic activity but they can not reveal if metabolic enzymes are activated and their substrates abundantly present. The present invention provides a stable isotope based dynamic metabolic profiling system with the purpose of obtaining dynamic metabolic substrate flow information through the pentose cycle, glycogen synthesis, tricarboxylic acid cycle, glycolysis, lactate synthesis, glutamate production, fatty acid synthesis and nucleic acid ribose and deoxyribose synthesis pathways simultaneously. It is therefore a comprehensive and dynamic technique based on precisely directed isotope labeling that can reveal specific metabolic pathway flux changes in disease and health. Further, the invention can be used to reveal the metabolic mechanisms of drug actions and that of natural/synthetic compounds in various disease treatment modalities and to improve metabolic engineering.

Features of this invention include:

1) The ¹³C stable isotope labeled metabolome allows not only the determination of substrate levels but also the determination of through which steps molecule synthesis pathways are linked;

2) A number of metabolic processes are simultaneously determined in the same cell system or organism;

3) The preferred synthesis steps that can be effectively targeted by new drugs (steps with large control coefficients) for individual metabolites can be predicted and determined;

4) The obtained metabolic profiles of diseases or drug actions can be compared, correlated and used to define disease states, and responses to gene manipulations, signaling events or drug treatments;

5) Early toxic effects of new compounds are readily revealed by the deterioration of isotope labeled carbon flow through life sustaining metabolic pathways; and

6) The direct metabolic effects of “silent genes”, which do not alter metabolite concentrations but synthesis pathways only for the same metabolite are revealed.

The dynamic metabolic profiling method of the invention involves a “smartly” labeled glucose substrate for the positional labeling of other metabolites in the cell during various metabolic steps. By analyzing the stable isotope labeled metabolome the method may reveal synthesis pathway specific metabolic adaptive changes in response to practically any condition in the environment, during health, disease or drug treatments. The dynamic and comprehensive stable isotope based metabolic profiling technique utilizes a broad yet specific approach for multiple pathway flux measurements and synthesis pathway activities based on the “smartly” labeled, nontoxic and stable isotope tracer. This specifically labeled substrate introduces “heavy” non-radiating carbons into specific positions of the carbon chain of several key intermediates, which then reveal vital information about pathway substrate flux and redistribution after recovery of the label from the product bio-molecules of the metabolome. The invention makes it possible to determine the concentrations of intermediate molecules as well as the ability to determine the dynamics of synthesis and turnover rates with accurate details of the contribution of specific synthetic reactions across metabolic networks.

The invention solves the basic problem of investigating metabolism in a dynamic, comprehensive and specific manner which can reveal actual metabolic pathway substrate utilization and distribution patterns, predict changes in metabolic enzyme activities and determine the metabolic end result of various genetic mutations, silent genes, disease processes, cell signaling events and chemical drug interventions.

The methodology of the invention possesses the advantage of being an interactive, comprehensive and treatment responsive metabolic screening tool for the drug industry and academic investigative processes.

The methodology of the invention uses isotope incorporation data to measure synthesis rates and molecule turnover rates while also providing the specificity of identifying particular pathways and making it possible to determine their contributions to previous and subsequent metabolic steps. The invention makes it possible to provide drug testing, drug target discovery or drug screening and apply the technology to studying basic biochemical events in primitive species including bacteria and yeast.

The present invention comprises, for example, the use of the stable [1,2-¹³C₂]glucose tracer for metabolic pathway analyses in cultures of mammalian cells, bacteria, virus hosting cells, phage hosting bacteria, tissue slices, perfused organs, living animals or humans. The method may further comprise applying such to individual pathway flux measurements, and analyzing the metabolome as whole or testing drugs. The invention provides a dynamic and comprehensive metabolic profiling technique by using specifically labeled glucose substrate isotopes which not only turn into intermediary metabolites but also produce mass isotopomers of these metabolites which can be separated, measured and quantitated using liquid and gas chromatography separation and mass spectrometry (GC/MS), other mass spectrometric analysis or nuclear magnetic resonance (NMR) instruments.

Preparation of samples for dynamic metabolic profiling may be carried out using methods described in the literature. The invention may comprise the design of the label system, its distribution and recovery from the same metabolites that have previously been isolated for metabolic profiling studies. However numerous metabolites can be isolated from the cell culture media, cell pellets or blood plasma simultaneously. This make it possible to measure interconnected metabolic pathway carbon substrate flow using one common substrate, glucose, for metabolic profiling drug action studies.

After treatments while incubating with the isotope labeled glucose substrate, the profiling study may begin with the separation of cell pellets, cell culture media, blood plasma/serum or body fluids. The cell culture media may then be used for lactate and glutamate analyses. Lactate is an abundant cell media component of the 3 carbon metabolite pool of the cell and it is used to determine the relative activity of pentose cycle glucose oxidation and recycling into glycolysis as the percent of substrate flux through glycolysis, also known as the Embden-Meyerhoff-Parnas pathway. Glutamate label accumulation represents tricarboxylic acid (TCA) cycle carbon substrate flow. Nucleic acid ribose and deoxyribose are used to determine cell viability (ribose) and cell proliferation (deoxyribose).

Cell cycle progression and the frequency of cell divisions are determined by the accumulation of ¹³C label into deoxyribose, while cell viability, apoptosis and necrosis are determined by the differential incorporation of ¹³C into ribose and deoxyribose. The major advantage of our label system introduced herein is that we can differentiate between glucose oxidation and nonoxidative ribose/deoxyribose synthesis during nucleic acid production in disease and health as well as during drug testing. Glycogen glucose represents glycogen synthesis, which can originate from activated glucose (direct glycogen synthesis) or indirectly from pentoses after glucose oxidation in the pentose cycle. Non-essentail fatty acids of the saturated and desaturated kinds indicate the rate of de novo fatty acid synthesis from glucose and the contribution of fatty acid synthase, chain elongase and desaturase to cell differentiation, hormone synthesis and drug effects.

The label system of the invention may be used to differentiate and characterize these pathways and their responses to drug treatments in a specific and effective manner in a simple series of labeling and drug treatment studies. The stable isotope label system does not interfere with drug effects and the effect of drugs can therefore be studied simultaneously in virtually all major interconnected metabolic pathways which also serve in energy production, cell proliferation, enzyme, hormone or specific metabolite synthesis pathways.

Although there are many tools and devices to analyze stable isotope labeled metabolites that include NMR or new mass spectrometry techniques, such as MALDI-TOF, which require different sample preparation methods and techniques, it may be that [1,2-¹³C₂]glucose provides the most comprehensive, effective and cost efficient label design for dynamic metabolic profiling purposes.

The invention makes it possible to track enzymatic modifications of the precisely labeled precursor molecule as it makes specific rearrangements in the positions and amounts of the stable isotope incorporated into subsequent bio-molecules throughout the metabolome. The rearrangements of labeled carbons within a molecule yield the dynamic history of that molecule from the precursor to the product and between, much the same way as if the labeled molecule had a “memory” imprinted with the steps it went through.

The method of the invention may be used to not only predict but exactly determine the metabolic steps involved in the formation of any glucose-based or glycogenic metabolite present in a living organism. This may include glycogenic nonessential amino acids, nucleic acid ribose/deoxyribose and their bases, TCA cycle metabolites, phosphorylated glycolytic products, pentose cycle intermediates, glycogen, lactate, glutamate and non-essential fatty acids of the saturated and unsaturated kinds. These metabolites can be used to determine substrate flow and metabolic activity through glycolysis, glycogen synthesis, TCA cycle, pentose cycle, fatty acid synthesis and specific amino acid synthesis rates in mammalian cells, organisms and hosts. Known metabolic cycles of living organisms that can specifically be characterized by the [1,2-¹³C₂]glucose isotope tracer and dynamic metabolic profiling are shown in FIG. 1.

The present invention comprises the introduction of a precisely labeled general precursor molecule with a harmless non-radiating stable isotope on specific carbon positions. The label is allowed to distribute and rearrange with existing molecules and substrate pools in the cell. The next steps are to sample these pools, recover the stable isotope labeled intermediates and determine the positional distribution of the label. From the mass isotopomer information obtained, the invention provides information in the metabolic pathway flux and enzyme activities changes, the effects of genes and signaling pathways on metabolism. This information can be used to provide a comparison between healthy and diseased tissues and allows for the evaluation of drug treatments and other therapeutic interventions.

These and other objects, advantages, and features of the invention will become apparent to those persons skilled in the art upon reading the details of the invention as more fully described below.

### BRIEF DESCRIPTION OF THE DRAWINGS

FIG. 1 is a schematic drawing of the overall metabolic networks of living organisms, the inter-connecting metabolic steps and key metabolites that are readily labeled by [1,2-¹³C₂]glucose as the tracer precursor.

FIG. 2 is a schematic drawing of the structure of a preferred embodiment of a labeled glucose molecule along with possible rearrangements of ¹³C in various metabolites of glycolysis using [1,2-¹³C₂]glucose as the single tracer.

FIG. 3 is a schematic drawing of the structure of the labeled compounds involved in the formation of [2,3-¹³C₂]lactate through the Embden-Meyerhoff-Parnas pathway.

FIG. 4 is a schematic drawing of the structure of compounds involved in the rearrangement of ¹³C in pentose cycle metabolites due to direct glucose oxidation.

FIG. 5 is a schematic drawing of the structure of compounds involved in formation of [1-¹³C]ribose-5P in the non-oxidative pentose cycle after glucose oxidation.

FIG. 6 is a schematic drawing of the structure of compounds involved in the formation of [1,2-¹³C₂]ribose through the non-oxidative reactions of the pentose cycle.

FIG. 7 is a schematic drawing of the structure of compounds involved in the formation of ¹³C labeled acetyl-CoA and glutamate through pyruvate dehydrogenase and pyruvate carboxylase in the TCA cycle.

### DETAILED DESCRIPTION OF PREFERRED EMBODIMENTS

Before the present invention is described, it is to be understood that this invention is not limited to molecules and specific method steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a labeled molecule” includes a plurality of such labeled molecules and reference to “the step” includes reference to one or more steps and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Invention in General

Molecules involved in cellular metabolism are labeled at particular known positions for the invention. The labeled molecules are tracked and analyzed at one or more points in time as they move through and between metabolic cycles. Once precise information is gathered on how a labeled molecule is changed and distributed in a known system (e.g. a system comprised of a particular type of cells in a known cell culture medium) the system is characterized. System characteristics may change by adding a compound being developed as a possible pharmaceutically active drug. The manner in which the drug alters the way the labeled molecule is acted on by the system can then be used for drug characterization purposes. The changes observed relative to control drug free cultures in the system may be used to provide valuable information on factors such as mechanism of action, safety and efficacy of the drug tested.

The method of the invention can be carried out with a number of different end results obtained. For example, the end result may be an evaluation of the effect of a compound such as a proposed pharmaceutically active drug on one or more metabolic pathways. The method is carried out by labeling precursor molecules which are preferably labeled with ¹³C isotope at a known position. The precursor molecule can be any molecule which normally contains a ¹²C. Further, 1, 2, 3, 4, 5, 6 or any number of ¹³C labels can be included within the precursor molecule. An example of a precursor molecule typically utilized in connection with the invention is a glucose molecule.

Once the precursor molecule such as the glucose molecule is labeled by having a ¹³C added in place of a ¹²C the precursor molecules are added to a changing test system. In terms of this system changing can be changing in any manner. However, it is typically a living system such as a cell, a group of cells in a cell culture or an animal which could be a human. Thus, changing does not mean that this system is completely different from one time to the next but rather that it is continuing to undergo biochemical reactions as are normally present within any living system.

After the labeled precursor molecules are added to the changing test system samples are extracted from the test system and molecules which incorporate the ¹³C label are analyzed. The molecules which are analyzed may be completely different molecules from the originally labeled ¹³C precursor molecules. The biochemical reactions of the changing test system may cause the ¹³C label to be added to other molecules, i.e. cause the normal carbon or ¹²C present in other molecules to be replaced by ¹³C. The analysis is carried out at a given point in time which can be referred to as a first point in time.

After the analysis is carried out the information is obtained and the obtained information is compared with information which may be obtained from a control system or compared to reference information which is previously obtained from a variety of sources including multiple control systems. In general, the control system is identical or substantially identical to the changing test system except that the test system has a single characteristic change. That single characteristic may, for example, be the addition of a compound which may be a proposed pharmaceutically active drug. The information is generally information such as changes in the molecular weight caused by the addition of the ¹³C molecule being analyzed. Such makes it possible to determine the position of the ¹³C within the molecule being analyzed. Thereby making it possible to determine the effects of the change such as changes caused by the pharmaceutically active drug on the system such as a change induced in a metabolic pathway of the system.

Preferably, samples are taken at multiple times which may be a second, a third, or fourth time which are each later in time from the first time and from each other. The samples taken at these different times are then analyzed and then compared with comparable information from a control system assayed at substantially the same points in time.

What the method of invention makes possible is the tracking of carbon atoms as they move through one or more different metabolic pathways of a changing system such as a cell culture. The precise position of the labeled carbon atom in a molecule can be tracked. The movement of that labeled carbon atom from one molecule to another at specific positions can reveal substantial amounts of information relating to the metabolic pathway of a living system. When that information is compared with a control system it is possible to obtain relatively precise information regarding the effect of a change such as an added proposed drug has on the system.

The system used can generally be any environment, which acts on and changes metabolic reactions and/or the molecule involved in such reactions. Thus, the system can be a chemical reaction, e.g. an enzymatically driven nucleotide replication reaction. At a more complex level the system can be a cell or cells in a cell culture medium. Any type of cell can be used including prokaryotic and eukaryotic cells, which may be alone or with viruses or phages. In particular, mammalian cells such as human cells can be used. Plant as well as animal cells can be used. The system is preferably contained in a manner so as to reduce or eliminate unwanted influences.

At a still more complex level the system may be a tissue culture comprised of plant or animal tissue. The tissue may be, for example, tissue from a particular organ, which may be acted upon by the drug. By examining the effects of the drug on the system at a molecular level and comparing such to the system in the absence of the drug and/or to other known systems a great deal of information on the safety and efficacy of the drug can be ascertained.

At a still more complex level the system may be a multi-cellular organism. The organism may be a plant or an animal including a human. In one embodiment the system is a transgenic, non-human animal genetically engineered to have or be capable of getting a disease generally associated with humans. Testing drugs in transgenic animals could always provide some information on the safety and efficacy of the drug but can provide substantially more information via the present invention.

The system can be human or animal tissue hosting bacteria or viruses in order to study the metabolic particulars of bacterial replication or virus assembly in the presence of any type of pharmaceutically active drug, e.g. oncology and antibiotics in particular.

The system can be plant cells or bacteria hosting phages in culture or in vivo in order to study phage replication and assembly in primitive organisms.

### Glucose Intermediates Produced

The changing pattern of distribution of ¹³C carbons from [1,2-¹³C₂]glucose in intracellular metabolic intermediates may be used to provide a measure of carbon flow toward the pentose cycle, glycolysis, direct glucose oxidation, TCA cycle and fatty acid synthesis, simultaneously. Metabolic profiling reveals specific flux changes in lactate, glutamate, nucleic acid ribose, palmitate and CO₂ during disease and health or during drug treatments or other interventions. Dynamic and comprehensive metabolic profiling thus indicates specific changes in glucose substrate utilization for macromolecule synthesis in living organisms, reveals the synthesis steps and provides information that can also be used for drug target development. The rationale for ¹³C labeling and interpretation of information gained by it during metabolic profiling are described below.

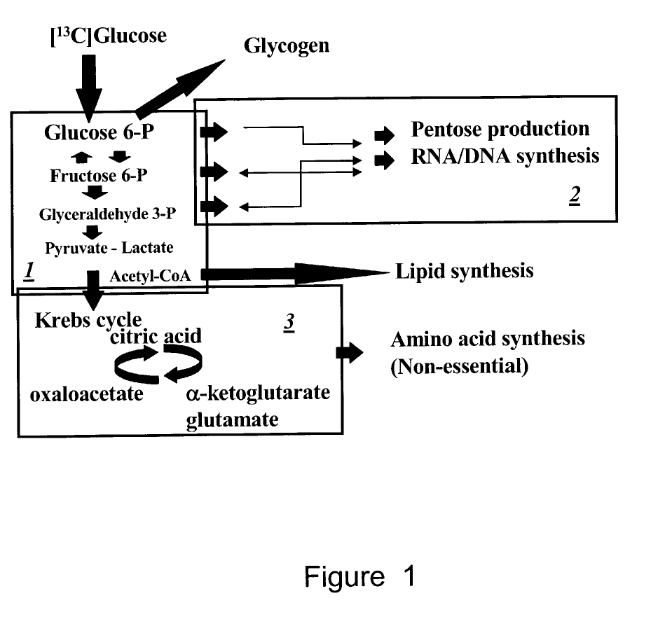

Glucose enters the cell as a broad and widely used substrate, or precursor of others, upon which it becomes activated (phosphorylated) as shown in FIG. 1. Glucose provides carbons for the synthesis of glycogen, pentoses, nucleotides, glycolysis intermediates, TCA cycle metabolites, fatty acids, lactate, amino acids and many other molecules not discussed here. Box 1 in FIG. 1 shows glycolysis, box 2 shows the pentose cycle oxidative and nonoxidative branches and box 3 shows the tricarboxylic acid (TCA) cycle. ¹³C labeled glucose readily enters these metabolic cycles and labels metabolite pools. By rearrangements of the ¹³C specific substrate flow information may be gathered by the invention. For example, ribose synthesis in the pentose cycle is possible through either the oxidative or the nonoxidative branches. Conventional label systems generally do not differentiate between the two branches, which produce chemically identical ribose. By the rearrangements of ¹³C in ribose the method of the invention can be used to differentiate between oxidative and nonoxidative pentose production which is a crucial metabolic process for nucleic acid synthesis, cell proliferation and cell differentiation.

In general, [1,2-¹³C₂]glucose metabolism produces four isotope-labeled intermediary metabolite species, also called mass isotopomers, m1: with one ¹³C substitution; m2: with two ¹³C substitutions; m3: with three ¹³C substitutions; and m4: with four ¹³C substitutions; which can reside in various positions in intermediary metabolites. These isotopomers are readily separated and measured using gas chromatography/mass spectrometry techniques described previously (Andrew, R. (2001) Clinical measurement of steroid metabolism. Best Pract Res Clin Endocrinol Metab 15: 1-16; Lee, W. N., Boros, L. G., Puigjaner, J., Bassilian, S., Lim, S., Cascante, M. (1998) Mass isotopomer study of the non-oxidative pathways of the pentose cycle with [1,2-¹³C₂] glucose. Am. J. Physiol. 274, E843-51; Lee, W. N., Edmond, J., Bassilian, S., Morrow, J. W. (1996) Mass isotopomer study of glutamine oxidation and synthesis in primary culture of astrocytes. Dev. Neurosci. 18, 469-77; Lee, W. N., Byerley, L. O., Bassilian, S., Ajie, H. O., Clark, I., Edmond, J., Bergner, E. A. (1995) Isotopomer study of lipogenesis in human hepatoma cells in culture: contribution of carbon and hydrogen atoms from glucose. Anal. Biochem. 226, 100-12).

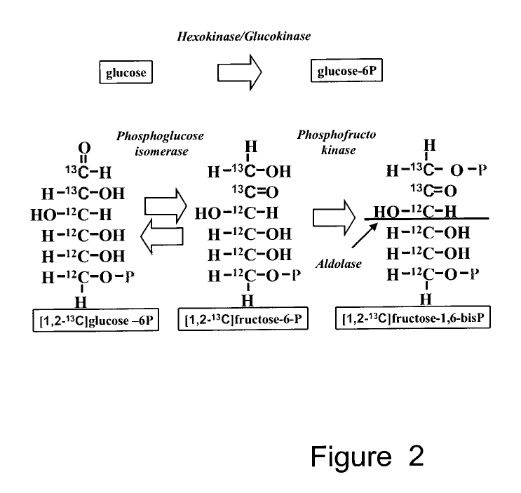

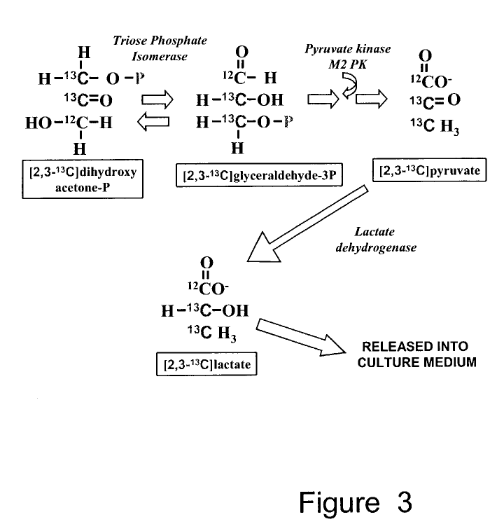

Lactate is the main three-carbon product of glycolysis and it is readily secreted into the cell culture medium. Accordingly, lactat can be utilized for the measurement of label incorporation into the three-carbon metabolite pool. The possible arrangements of ¹³C labels from [1,2-¹³C₂]glucose to lactate through glycolysis are shown in FIG. 2 and FIG. 3.

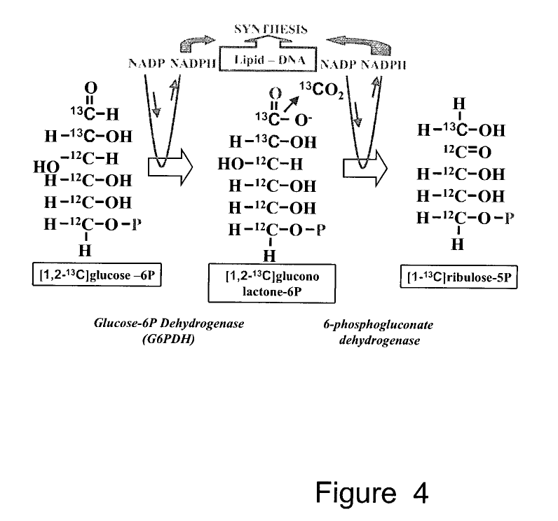

Glucose oxidation through the pentose cycle on the other hand results in a loss of the first ¹³C of glucose that is shown in FIG. 4. During glucose oxidation ¹³CO₂ is also released which reflects glucose utilization for energy production in the pentose and TCA cycles. During metabolic profiling the method of the invention makes it possible to determine not only the amount of ¹³C isotope accumulation but also the positions of ¹³C labeled carbons within lactate.

Those skilled in the art reading this disclosure will recognize that the ratio between m1 (recycled lactate from oxidized glucose via the oxidative branch of pentose cycle) and m2 (lactate produced by the Embden-Meyerhof-Parnas glycolytic pathway) is indicative of the activity of G6PDH and glucose recycling in the pentose cycle. A detailed description of the reactions and calculations can be found elsewhere (Lee, W. N., Boros, L. G., Puigjaner, J., Bassilian, S., Lim, S., Cascante, M. (1998) Mass isotopomer study of the non-oxidative pathways of the pentose cycle with [1,2-¹³C₂]glucose. Am. J. Physiol. 274, E843-51). Disease processes and drug treatments that affects direct glucose oxidation or glycolytic flux is expected to alter glucose label rearrangement in lactate.

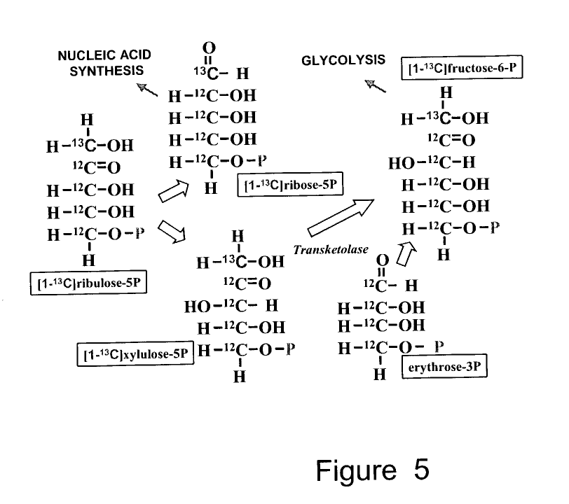

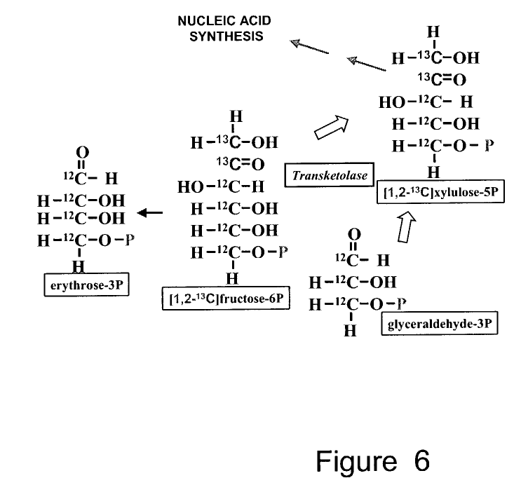

Ribose and deoxyribose are the building blocks of nucleotides and therefore ¹³C incorporation from glucose into RNA ribose or DNA deoxyribose indicates changes in nucleic acid synthesis rates through the respective branches of the pentose cycle. Singly labeled ribose molecules on the first carbon position (m1) represent ribose that is produced by direct glucose oxidation through G6PDH (FIG. 4). This ribose can either be incorporated into nucleic acid or returned to glycolysis as shown in FIG. 5. The alternative pathway for ribose synthesis is through the non-oxidative steps of the pentose cycle using glycolytic metabolites (FIG. 6). There is no net carbon loss throughout the non-oxidative steps of the pentose cycle; therefore, ribose molecules labeled on the first two carbon positions with ¹³C (m2) represent nucleic acid ribose synthesis through the non-oxidative route. The ratio between m1, m2, m3 and m4 of nucleic acid ribose/deoxyribose closely reflects the involvement of glucose oxidation and non-oxidative ribose synthesis in tumor cells. These reactions are effectively modulated by diseases and by various treatment modalities during de novo nucleic acid synthesis and cell growth. Anti-cancer drugs which restrict new cell growth and antibiotics which restrict bacterial growth can accordingly, be monitored via methods of the invention.

¹³CO₂ release is a reliable marker of glucose oxidation (FIG. 4). ¹³CO₂ production from [1,2-¹³C]glucose takes place in both the pentose and TCA cycles and it is measured as part of the metabolic profiling processes to determine the rate of glucose oxidation in response to various drug therapies. Decreased glucose oxidation with increased glucose uptake is always a reliable sign of increased anabolism as seen in transformed cells.

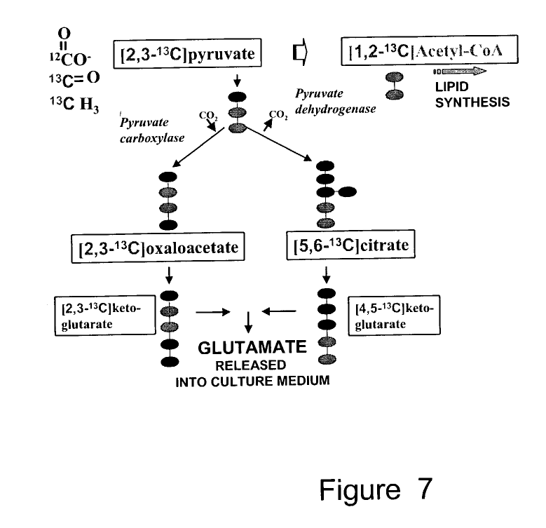

Glutamate, a non-essential amino acid, is partially produced from mitochondrial α-ketoglutarate, which is a central intermediate of the TCA cycle. Glutamate is readily released into the culture medium after synthesis, which represents one of the routes for glucose carbon utilization. Therefore, label incorporation from glucose into glutamate is a good indicator of TCA cycle anabolic metabolism for amino acid synthesis instead of glucose oxidation (FIG. 7).

Fatty acid synthesis is also strongly dependent on glucose carbons through the formation of acetyl-CoA via pyruvate dehydrogenase. The incorporation of ¹³C from [1,2-¹³C₂]glucose gives key information about the fraction of de novo lipogenesis in mammalian cells and about glucose carbon contribution to acetyl-CoA for fatty acid synthesis (FIG. 7). Many diseases and treatment modalities alter fatty acid synthesis, and changes in the flow of carbon toward fatty acid synthesis are important in cell growth control, differentiation, enzyme/hormone synthesis and new receptor formation.

The study of dynamic metabolic profiles using stable isotopes in cell cultures or in vivo reveals how the signaling and genetic events translate into metabolic processes, and also how substantially metabolic pathway flux changes influence cell growth. Effective therapeutics and drugs will alter carbon substrate flow in metabolic pathways in a desired manner, which can be reveled using the stable isotope based dynamic metabolic profiling technique of the invention. Therefore dynamic metabolic profiling is an excellent tool for screening potential new drugs to treat diseases.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### ¹³C Labeled Glucose—Control/Test Systems

The details of how the invention can be carried out can be better understood by reference to the figures. For example, FIG. 2 shows the structure of a preferred embodiment of a labeled glucose molecule along with possible rearrangements of ¹³C in various metabolites of glycolysis using [1,2-¹³C₂]glucose as the single tracer. Glucose activation via hexokinase/glucokinase and the formation of fructose-1,6-bis phosphate maintain the ¹³C labeled carbons in the 1^{st} and 2^{nd} positions. ¹³C-labeled carbon positions derived from [1,2-¹³C₂]glucose are shown by the “13” superscript, while ¹²C native-labeled carbon positions are shown by the “12” superscript. Participating enzymes are italicized in all of the figures. Thus, a single version of the invention can be carried out by creating two separate cell culture systems. The first system is a control system which includes the ¹³C labeled glucose which may be [1,2-¹³C₂] glucose. The control system is provided with a desired environment including temperature, pH, and nutrients and samples are extracted and examined at different points (e.g. 1st, 2nd, 3rd etc.) in time to determine how the system acts on the labeled glucose. The second system is substantially identical to the first system except that a change is made e.g. a drug is added. Samples are then extracted and examined at substantially the same times and in substantially the same ways as per the control system. Then, the results of the control and test system are compared to determining if and how the change (e.g. the drug) affected the test system.

### Example 2

### ¹³C Label at Many Positions—Standard/Test Systems

In addition to labeling glucose as shown in FIG. 2, it is possible to label glucose at other positions and/or to label other molecules such as [2,3-¹³C₂] dihydroxy acetone-P or to continue to track the molecule of [2,3-¹³C₂]dihydroxy acetone-P created in the reaction shown in FIG. 2. FIG. 3 shows the structure of the labeled compounds involved in the formation of [2,3-¹³C₂]lactate through the Embden-Meyerhoff-Parnas pathway. The production of three-carbon metabolites by aldolase (as shown in FIG. 2), glyceraldehyde and dihdroxy acetone phosphates transfers the labeled carbons into the 2^{nd} and 3^{rd} positions of glyceraldehyde. There are no subsequent positional changes in terms of ¹³C labeling by triose phosphate isomerase in the three-carbon metabolite pool that undergoes glycolysis, resulting in the release of lactate. Thus, the method of the invention can be carried out as described about in Example 1. Those skilled in the art will understand that the control system may be unnecessary provided a standard or reference has been established in some manner. For example, the control may have been repeatedly carried out so that the results are known and those known results are used to create a standard or reference that can be compared to any desired test system. The more typical and generalized the system the easier it is to eliminate the control via an established standard. Alternatively, highly unique systems are more likely to require a control system as no standard or reference data have been established for purposes of comparison.

### Example 3

### Pentose Cycle Metabolites

The labeled glucose as shown in FIG. 2 can be acted on differently as the reactions of FIG. 4 show. FIG. 4 shows the structure of compounds involved in the rearrangement of ¹³C in pentose cycle metabolites due to direct glucose oxidation. The loss of the first labeled carbon of glucose due to direct oxidation produces ribubose molecules that are labeled only on the first position with ¹³C. During the oxidation of glucose ¹³CO₂ is released, which can easily be detected using isotope ratio mass spectrometry (IRMS). Reducing equivalent NADP⁺ is also produced that can be used in lipid synthesis, DNA nucleotide production or to maintain reductive/oxidative reactions throughout metabolism. The invention is particularly useful in monitoring the effects of drugs on these biochemical processes—using methods a described in Examples 1 and 2 above.

### Example 4

### Nucleic Acid Synthesis

The [1-¹³C]ribulose-5P molecule shown on the far right of FIG. 4 can be produced and labeled. Alternatively, the molecule can be tracked through one or more additional reactions. The molecule on the far right of FIG. 4 is on the far left of FIG. 5 which shows the structure of compounds involved in the formation of [1-¹³C]ribose-5P in the non-oxidative pentose cycle after glucose oxidation. The nonoxidative steps of the pentose cycle generate a number of intermediates that can be used for nucleic acid synthesis (ribose-5P, as seen in proliferating cells) or recycled back to glycolysis (glyceraldehydes-P and fructose-P, as seen in nonproliferating/resting cells). Monitoring nucleic acid synthesis provides information on cell growth/proliferation. Such information is used, for example, the test anti-cancer and antibiotic drugs as per Example 1 and 2 above.

### Example 5

### Monitoring Rapid Cell Proliferation

Nucleic acid synthesis is essential for cell replication and FIG. 6 shows the structure of compounds involved in the formation of [1,2-¹³C₂]ribose through the nonoxidative reactions of the pentose cycle. Rapidly proliferating cells are able to synthesize ribose-5P via non-oxidative pentose cycle reactions. This process allows the unrestrained production of ribose-5P, independent of available NADP, a phenomenon observed in response to cell transforming agents. Increased non-oxidative synthesis of ribose from glucose deprives mammalian cells of reducing equivalents. Although a great proliferating potential is engendered, reductive synthesis, differentiation, normal cell morphology and functions are diminished. Systems of rapidly proliferating cells (e.g. malignant tumor cells) can be established and used per the invention to test drugs as per Examples 1 and 2 above.

### Example 6

### The TCA Cycle

A compound such as pyruvate can also be labeled as shown FIG. 7 which shows [2,3-¹³C₂]pyruvate. FIG. 7 shows the structure of compounds involved in the formation of ¹³C labeled acetyl-CoA and glutamate through pyruvate dehydrogenase and pyruvate carboxylase in the TCA cycle. Glucose carbons readily label TCA cycle metabolites and fatty acids because the first two carbons of glucose form the acetate molecules that enter the TCA cycle and lipid synthesis pathways. Those skilled in the art, reading this disclosure will recognize the significance of using the methodology of the invention to monitor the effect of a compound such as a proposed pharmaceutically active drug on the TCA cycle. The TCA cycle can be effected by drugs or other factors and that effect can be determined via the invention as per Examples 1 and 2 above.

The examples 1-6 described above and schematically shown in FIGS. 2-7 demonstrate that the invention can be used in a wide variety of situations. Specifically, different molecules can be labeled and tracked for different periods of time through different metabolic cycles. It is preferable to label molecules that are well known to be acted on in a particular manner in a well-known and well characterized metabolic cycle. Thus, glucose carbons are generally labeled and monitored in known biochemical reactions. However, those skilled in the art, reading this disclosure will recognize the applicability of the invention to a wide range of molecules, reactions and systems.

### Example 7

### Informational Database

Genetic and signaling events in cells translate into metabolic changes that determine the function and phenotype of the cell. Changes in genetic, signaling and protein synthesis pathways can readily be revealed using molecular and proteomics technologies. The dynamic metabolic profiling technology provided here supplements these existing technologies by investigating changes through a specific stable isotope labeled metabolome. The invention can be carried out in a manner so as to develop a large database of the dynamic metabolome of various cell types. The information is then searched and used during drug design and target discovery in disease and health as a database, where sufficient patterns and pathway flux profiles are stored.

As the method of the invention is used and accepted it is expected to become the main metabolic profiling method for industrial and academic drug target design and new drug discovery processes. The invention makes it possible to set up “dynamic metabolic profiles” substrate utilization and distribution databases for various disease processes, signaling mechanisms, gene mutations and drug actions. This database can be searched for matching metabolic profiles by the industry or academic community in order to determine certain expected drug effects, signaling mechanisms and genetic events.

The metabolic profiles induced by cell transforming agents such as transforming growth factor beta (TGF-beta) and the organophosphate pesticide isofenphos have been determined. These profiles indicate intense nucleic acid ribose synthesis through the non-oxidative steps of the pentose cycle and increased cell proliferation rates (Cancer Research and Leukemia Research articles).

The metabolic profiles of anticancer compounds such as Avemar and Gleevec indicate that effective tumor growth control can be achieved when glucose activation, oxidative and non-oxidative ribose synthesis from glucose are inhibited. Further, increased fatty acid synthesis increases cell differentiation in response to Avemar treatment. These metabolic profiles can already be used as part of the stable isotope labeled metabolome database for additional drug effects and signaling mechanisms.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method, comprising the steps of:
adding ¹³C labeled molecules to a test system which test system acts on the molecules and which ¹³C label changes molecule weights of molecules which the label is added to;
analyzing ¹³C labeled molecules in the system after the system has acted on the ¹³C labeled molecule added to the system and obtaining information from the analyzing;
comparing the information obtained with information on a known system.

2. The method of claim 1, wherein the known system is a control system substantially identical to the test system except for a compound added to the test system.

3. The method of claim 1, wherein the information obtained from analyzing is of molecular weight of molecules incorporating a ¹³C label.

4. The method of claim 1, wherein the information obtained from the analyzing is of carbon positions of molecules incorporating a ¹³C label.

5. The method of claim 1, wherein the ¹³C labeled molecules are ¹³C labeled glucose.

6. The method of claim 5, wherein the labeled glucose molecules are chosen from [1,2-¹³C₂] glucose, [1,2,5,6-¹³C₄]glucose and [5,6-¹³C₂]glucose.

7. The method of claim 1, wherein the system comprises a living cell.

8. The method of claim 1, wherein the system comprises a plurality of living cells in a cell culture.

9. The method of claim 1, wherein the system comprises living tissue.

10. The method of claim 1, wherein the system comprises a multi-cellular organism.

11. The method of claim 1, wherein the system is chosen from bacteria and plant cells.

12. The method of claim 1, wherein the system comprises bacteria hosting phage.

13. The method of claim 1, wherein the system comprises cells hosting an infection chosen from viruses and virus particles.

14. The method of claim 1, comprising:
separating the labeled molecules from the system after changes in molecular weight to newly synthesized molecules have occurred.

15. The method of claim 14, wherein the separating is carried out by a means chosen from centrifugation, physical/chemical purification, chemical derivatization, liquid chromatography and gas chromatography.

16. The method of claim 1, wherein the analyzing is carried out by spectrometry.

17. The method of claim 16, wherein the spectrometry is selected from mass spectrometry and nuclear magnetic resonance.

18. The method of claim 1, further comprising:
adding a pharmaceutically active drug to the test system.

19. The method as claimed in claim 18, wherein the control system is substantially the same system as the test system to which the ¹³C labeled molecules are added, absent the pharmaceutically active drug.

20. A method of determining the specific metabolic steps involved in the formation of a glucose based metabolite of a living organism, comprising the steps of:
adding ¹³C labeled glucose to a living cellular system;
allowing the system to act on the labeled glucose for a given period of time under known conditions;
separating ¹³C labeled molecules away from the system;
analyzing the separated ¹³C labeled molecules; and
comparing analysis results to analysis results obtained from a known system.

21. The method of claim 20, wherein the living cellular system is a known cell culture grown on known cellular nutrients.

22. The method of claim 20, wherein the living cellular system is a non-human transgenic animal.

23. The method of claim 22, wherein the transgenic animal is a transgenic mouse.

24. The method of claim 20, wherein the animal is a genetically normal wild type animal.

25. The method of claim 20, wherein the living system is a human.

26. The method of claim 20 wherein the known system is a control system.

27. The method of claim 26, wherein the known system is a reference system.

28. A method, comprising the step of:
adding ¹³C labeled molecules to a system which ¹³C label replaces ¹²C in molecules increasing the molecular weight of the molecules where the ¹³C replaces a ¹²C;
analyzing molecules incorporating ¹³C labels to determine changes to molecular weights relative to when the molecule was comprised of ¹²C;
analyzing ¹³C labeled molecules to determine the positions of ¹²C and ¹³C labeled carbons;
comparing determined changes to molecular weights and ¹³C labeled carbon positions in control versus a drug treated system in order to reveal specific drug action on molecules of the system.

29. The method of claim 28, wherein the comparing is a comparing of changes to molecular weights and ¹³C labeled carbon positions in an organism chosen from a bacteria, cell, virus and phage to reveal metabolic pathways involved in the assembly of a progeny of the organism.

30. A method, comprising the steps of:
labeling precursor molecules with a ¹³C isotope at a known position;
adding the labeled precursor molecules to a changing test system;
analyzing molecules in the test system which molecules have incorporated the ¹³C label, wherein the analyzing is carried out at a first point in time;
comparing information obtained from the analyzing with information chosen from a control system information and reference information.

31. The method of claim 30, wherein the comparing of information is used to determine how a metabolic pathway of the test system is changed relative to the control system information or reference information.

32. The method of claim 30 further comprising:
analyzing molecules in the test system which have incorporated the ¹³C label at a second point in time after the first point in time.
